(19) United States
(12) Patent Application Publication
(10) Pub. No.: US 2006/0229254 A1
(43) Pub. Date: Oct. 12, 2006

(54) Compounds having prolyl oligopeptidase inhibitory activity

(76) Inventors: Jukka Gynther, Kuopio (FI); Erik Wallen, Kuopio (FI); Elina Jarho, Kuopio (FI); Pekka Mannisto, Helsinki (FI); Markus Forsberg, Kuopio (FI); Antti Poso, Kuopio (FI); Johannes Christiaans, Utrecht (NL); Jarkko Venalainen, Kuopio (FI); Jouko Vepsalainen, Kuopio (FI); Taija Saarinen, Helsinki (FI); Tomi Jarvinen, Kuopio (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/541,387

(22) PCT Filed: Jan. 2, 2004
(86) PCT No.: PCT/FI04/00001

§ 371 Date: May 9, 2006

(30) Foreign Application Priority Data
Jan. 3, 2003 (FI) 20030014

(51) Int. Cl. A61K 31/4025; A61K 38/04; C07K 5/04; C07D 403/02

(52) U.S. Cl. 514/19; 514/422; 548/518

(57) ABSTRACT

A compound of formula (I), 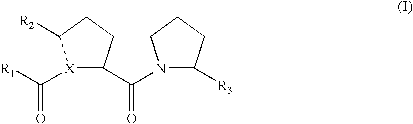
wherein X, R₁, R₂ and R₃ are as defined in the disclosure, or a pharmaceutically acceptable salt or ester thereof, useful as a prolyl oligopeptidase inhibitor. The compounds can be used for the treatment of diseases or conditions where prolyl oligopeptidase inhibitors are indicated to be effective, for example for the treatment of neurodegenerative diseases, such as Alzheimer's disease and senile dementia.

### FIELD OF THE INVENTION

The present invention relates to new prolyl oligopeptidase inhibitors, and to their pharmaceutically acceptable salts and esters thereof, as well as to pharmaceutical compositions containing them and to their use as a medicament.

### BACKGROUND OF THE INVENTION

Prolyl oligopeptidase (EC, 3.4.21.26) (POP), also known as prolyl endopeptidase, is the only serine protease that catalyses the hydrolysis of peptides at the C-terminal side of L-proline residues. It is widely distributed in mammals and can be purified from various organs, including the brain.

The enzyme plays an important role in the breakdown of proline-containing neuropeptides related to learning and memory functions (Wilk, S., Life Sci., 1983, 33, 2149-2157; O'Leary, R. M., O'Connor, B., J. Neurochem., 1995, 65, 953-963). Compounds capable of inhibiting prolyl oligopeptidase are effective for preventing experimental amnesia induced by scopolamine in rats, inferring that prolyl oligopeptidase inhibitors have functions in the alleviation of mnemonic dysfunctions (Yoshimoto, T., Kado, K., Matsubara, F., Koryama, N., Kaneto, H., Tsuru, D., J. Pharmacobio-Dyn., 1987, 10, 730-735).

In recent years it has been found that β-amyloid protein shows neurotoxic action in in vitro and in vivo experiments and that it may play an important role in the pathogenesis of Alzheimer's disease. In view of the hypothesis that substance P can suppress neurotoxic action of β-amyloid protein (Kowall, N. W., Beal, M. F., Busciglio, J., Duffy, L. K., Yankner, B. A., Proc. Natl. Acad. Sci. USA, 1991, 88, 7247-7251), it is speculated that prolyl oligopeptidase inhibitors that inhibit also metabolism of substance P will be discovered to be an effective drug for the treatment of Alzheimer's disease.

### SUMMARY OF THE INVENTION

The present invention relates to novel prolyl oligopeptidase inhibitors having the general formula (I):

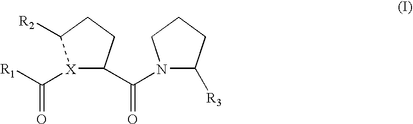

wherein in the formula, X is N or C;

the dotted line represents a single or a double bond;

R₁ is:

a straight or branched, unsubstituted or substituted alkyl chain having 1 to 10 carbon atoms,

a straight or branched, unsubstituted or substituted alkenyl chain having 2 to 10 carbon atoms,

a 3 to 7 membered, saturated or unsaturated, unsubstituted or substituted carbocyclic ring,

a 3 to 7 membered, saturated or unsaturated, unsubstituted or substituted heterocyclic ring,

a substituted or unsubstituted alkyl or alkenyl group as defined above incorporating as a group member a substituted or unsubstituted carbocyclic ring or a heterocyclic ring as defined above,

hydroxy, lower alkoxy, aryloxy, aryl lower alkoxy, amino, amino lower alkyl, lower alkyl amino, aryl amino or aryl lower alkyl amino, wherein the said alkyl, aryl or amino subgroups are unsubstituted or substituted;

R₂ is:

H,

a straight or branched, unsubstituted or substituted alkyl chain having 1 to 10 carbon atoms,

a straight or branched, unsubstituted or substituted alkenyl chain having 2 to 10 carbon atoms,

or a straight or branched, unsubstituted or substituted alkynyl chain having 2 to 10 carbon atoms;

R₃ is:

H, cyano, hydroxy, oxo, halogen, lower alkyl, lower alkoxy, aryl, aryloxy, aryl lower alkoxy, amino, lower alkyl amino, aryl amino, aryl lower alkyl amino, cycloalkyl or heterocycle, wherein the said alkyl subgroups are unsubstituted or substituted,

or R₃ is COOR⁴, COR⁴, CR⁴(OR⁵)₂ or COCH₂OR⁶, wherein R⁴ is H, lower alkyl, lower alkenyl, cycloalkyl, cycloalkenyl, heterocycle, aryl, amino, lower alkyl amino, aryl amino or lower alkyl amino, wherein the said lower alkyl are unsubstituted or substituted, R⁵ is lower alkyl, lower alkenyl, cycloalkyl, cycloalkenyl, aryl or aralkyl and R⁶ is lower acyl or halogen;

provided, that

a) when X is N, the dotted line represents a single bond and R₂ is not H;

b) when X is C, the dotted line represents a double bond and R₂ is H.

The present invention also relates to the pharmaceutically acceptable salts and esters of the compounds of the formula (I). Pharmaceutically acceptable salts, e.g. acid addition salts with both organic and inorganic acids are well known in the field of pharmaceuticals. Non-limiting examples of these salts include chlorides, bromides, sulfates, nitrates, phosphates, sulfonates, formates, tartrates, maleates, citrates, benzoates, salicylates and ascorbates. Pharmaceutically acceptable esters, when applicable, may be prepared by known methods using pharmaceutically acceptable acids that are conventional in the field of pharmaceuticals and that retain the pharmacological properties of the free form. Non-limiting examples of these esters include esters of aliphatic or aromatic alcohols, e.g. methyl, ethyl, propyl, isopropyl, butyl, isobutyl, sec-butyl and tert-butyl esters.

A further object of the invention is a pharmaceutical composition containing at least one pharmaceutically acceptable diluent, carrier, and/or excipient, as well as a therapeutically effective amount of a compound of the formula (I) as the active agent. Still a further object of the invention is the use of the compounds of the formula (I) as a prolyl oligopeptidase inhibitor, for example in the treatment of neurodegenerative diseases, such as for Alzheimer's disease, and senile dementia, as well as for improving learning and memory functions. Furthermore, a method for the treatment of a disesase or the enhancement of a condition where prolyl oligopeptidase inhibitors are indicated to be useful, e.g. a method for the treatment of neurodegenerative diseases, and/or for the improvement of learning and memory functions, is provided. In such a method a therapeutically effective amount of a compound of the invention is administered to a subject in need of such treatment. The use of the compounds of the invention for the manufacture of a medicament to be used for the above indication is also provided.

The compounds of formula (I), as well as the pharmaceutically acceptable salts and esters thereof, are referred to below as the compounds of the invention, unless otherwise indicated.

The invention includes within its scope all the possible stereoisomers of the compounds of formula (I), including geometric isomers, e.g. Z and E isomers (cis and trans isomers), and optical isomers, e.g. diastereomers and enantiomers. Furthermore, the invention includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g. enantiomers, from the mixture thereof the conventional resolution methods, e.g. fractional crystallisation, may be used.

### DETAILED DESCRIPTION OF THE INVENTION

In the above-mentioned formula (I), the symbols have the following meanings:

X represents N or C.

The dotted line represents a single or a double bond.

A straight or branched alkyl chain in the meaning of R₁ has 1 to 10 carbon atoms. Such a group is unsubstituted or substituted with 1 to 3 substituent(s) each independently being COOR⁴, COR⁴, CR⁴(OR⁵)₂, COCH₂OR⁶, cyano, hydroxy, oxo, halogen, lower alkoxy, aryl, aryloxy, aryl lower alkoxy, nitro, amino, lower alkyl amino, aryl amino, aryl lower alkyl amino, cycloalkyl or heterocycle, wherein R⁴ is H, lower alkyl, lower alkenyl, cycloalkyl, cycloalkenyl, heterocycle, aryl or aralkyl, R⁵ is lower alkyl, lower alkenyl, cycloalkyl, cycloalkenyl, aryl or aralkyl and R⁶ is H, lower alkyl, lower acyl or halogen.

A straight or branched alkenyl chain in the meaning of R₁ has 2 to 10 carbon atoms. Such a group is unsubstituted or substituted with 1 to 3 substituent(s) as defined for the alkyl group above.

A carbocyclic ring in the meaning of R₁, or incorporated as a chain member in the alkyl or alkenyl group, is a saturated or unsaturated 3 to 7 membered ring with only carbon atoms in the ring. Such a group is unsubstituted or substituted with 1 to 3 substituent(s) each independently being lower alkyl or as defined for the alkyl group above.

A heterocyclic ring in the meaning of R₁, or incorporated as a chain member in the alkyl or alkenyl group, is a saturated or unsaturated 3 to 7 membered heterocyclic ring containing 1 to 3 heteroatom(s) selected from a nitrogen atom, an oxygen atom and/or sulphur atom. The heterocyclic group R₁ is unsubstituted or substituted with 1 to 3 substituent(s) each independently being lower alkyl or as defined for the alkyl group above.

When R₁ is hydroxy, lower alkoxy, aryloxy, aryl lower alkoxy, amino, amino lower alkyl, lower alkyl amino, aryl amino or aryl lower alkyl amino, the said alkyl, aryl or amino subgroups are unsubstituted or substituted with 1 to 3 substituent(s) each independently being lower alkyl or as defined for the alkyl group above.

A straight or branched alkyl chain in the meaning of R₂ has 1 to 10 carbon atoms. Such a group is unsubstituted or substituted with 1 to 3 substituent(s) each independently being hydroxy, oxo, lower alkoxy, amino, lower alkyl amino, halogen, carboxyl or lower acyl.

A straight or branched alkenyl chain in the meaning of R₂ has 2 to 10 carbon atoms. Such a group is unsubstituted or substituted with 1 to 3 substituent(s) as defined for the alkyl group, in the meaning of R₂, above.

A straight or branched alkynyl chain in the meaning of R₂ has 2 to 10 carbon atoms. Such a group is unsubstituted or substituted with 1 to 3 substituent(s) as defined for the alkyl group, in the meaning of R₂, above.

When R₃ is H, cyano, hydroxy, oxo, halogen, lower alkyl, lower alkoxy, aryl, aryloxy, aryl lower alkoxy, amino, lower alkyl amino, aryl amino, aryl lower alkyl amino, cycloalkyl or heterocycle, the said alkyl subgroups are unsubstituted or substituted with 1 to 3 substituent(s) as defined for the alkyl group, in the meaning of R₁, above.

When R₃ is COOR⁴, COR⁴, CR⁴(OR⁵)₂ or COCH₂OR⁶, R⁴ is H, lower alkyl, lo alkenyl, cycloalkyl, cycloalkenyl, heterocycle, aryl, amino, lower alkyl amino, aryl amino or lower alkyl amino, wherein the said lower alkyl is unsubstituted or substituted with 1 or 2 substituent(s) each independently being cyano, hydroxy, oxo, halogen, lower alkoxy, aryl, aryloxy, aryl lower alkoxy, amino, lower alkyl amino, aryl amino, aryl lower alkyl amino, cycloalkyl or heterocycle, R⁵ is lower alkyl, lower alkenyl, cycloalkyl, cycloalkenyl, aryl or aralkyl and R⁶ is lower acyl or halogen.

In the above-mentioned formula (I), the symbols have the meanings as described with the provisos that

a) when X is N, the dotted line represents a single bond and R₂ is not H;

b) when X is C, the dotted line represents a double bond and R₂ is H.

The compounds of the invention may be converted, if desired, into their pharmaceutically acceptable salt or ester form using methods well known in the art.

A possible subgroup of the compound of formula (I) is a compound wherein

X is N;

the dotted line represents a single bond;

R₁ is:

a straight or branched alkyl chain having 1 to 10 carbon atoms unsubstituted or substituted with 1 to 3 substituent(s) each independently being COOR⁴, COR⁴, CR⁴(OR⁵)₂, COCH₂OR⁶, cyano, hydroxy, oxo, halogen, lower alkoxy, aryl, aryloxy, aryl lower alkoxy, nitro, amino, lower alkyl amino, aryl amino, aryl lower alkyl amino, cycloalkyl or heterocycle, wherein R⁴ is H, lower alkyl, lower alkenyl, cycloalkyl, cycloalkenyl, heterocycle, aryl or aralkyl, R⁵ is lower alkyl, lower alkenyl, cycloalkyl, cycloalkenyl, aryl or aralkyl and R⁶ is H, lower alkyl, lower acyl or halogen, a straight or branched alkenyl chain having 2 to 10 carbon atoms unsubstituted or substituted with 1 to 3 substituent(s) as defined for the alkyl group above, a 3 to 7 membered, saturated or unsaturated, carbocyclic ring unsubstituted or substituted with 1 to 3 substituent(s) each independently being lower alkyl or as defined for the alkyl group above,

a 3 to 7 membered, saturated or unsaturated, heterocyclic ring unsubstituted or substituted with 1 to 3 substituent(s) each independently being lower alkyl or as defined for the alkyl group above,

a substituted or unsubstituted alkyl or alkenyl group as defined above incorporating as a group member a substituted or unsubstituted carbocyclic ring or a heterocyclic ring as defined above,

hydroxy, lower alkoxy, aryloxy, aryl lower alkoxy, amino, amino lower alkyl, lower alkyl amino, aryl amino or aryl lower alkyl amino, wherein the said alkyl, aryl or amino subgroups are unsubstituted or substituted with 1 to 3 substituent(s) each independently being lower alkyl or as defined for the alkyl group above;

R₂ is:

a straight or branched alkyl chain having 1 to 10 carbon atoms unsubstituted or substituted with 1 to 3 substituent(s) each independently being hydroxy, oxo, lower alkoxy, amino, lower alkyl amino, halogen, carboxyl or lower acyl,

a straight or branched alkenyl chain having 2 to 10 carbon atoms unsubstituted or substituted with 1 to 3 substituent(s) as defined for the alkyl group, in the meaning of R₂, above,

or a straight or branched alkynyl chain having 2 to 10 carbon atoms unsubstituted or substituted with 1 to 3 substituent(s) as defined for the alkyl group, in the meaning of R₂, above;

R₃ is:

H, cyano, hydroxy, oxo, halogen, lower alkyl, lower alkoxy, aryl, aryloxy, aryl lower alkoxy, amino, lower alkyl amino, aryl amino, aryl lower alkyl amino, cycloalkyl or heterocycle, wherein the said alkyl subgroups are unsubstituted or substituted with 1 to 3 substituent(s) as defined for the alkyl group, in the meaning of R₁, above, or R₃ is COOR⁴, COR⁴, CR⁴(OR⁵)₂ or COCH₂OR⁶, wherein R⁴ is H, lower alkyl, lower alkenyl, cycloalkyl, cycloalkenyl, heterocycle, aryl, amino, lower alkyl amino, aryl amino or lower alkyl amino, wherein the said lower alkyl is unsubstituted or substituted with 1 or 2 substituent(s) each independently being cyano, hydroxy, oxo, halogen, lower alkoxy, aryl, aryloxy, aryl lower alkoxy, amino, lower alkyl amino, aryl amino, aryl lower alkyl amino, cycloalkyl or heterocycle, R⁵ is lower alkyl, lower alkenyl, cycloalkyl, cycloalkenyl, aryl or aralkyl and R⁶ is lower acyl or halogen, or a pharmaceutically acceptable salt or ester thereof; for example

wherein R₁ is

a straight or branched alkyl chain having 1 to 5 carbon atoms unsubstituted or substituted with 1 or 2 substituent(s) each independently being hydroxy, halogen, lower alkoxy, aryl, aryloxy, aryl lower alkoxy, amino, lower alkyl amino, aryl amino, aryl lower alkyl amino, cycloalkyl or heterocycle,

a 3 to 7 membered, saturated or unsaturated, carbocyclic ring unsubstituted or substituted with 1 or 2 substituent(s) each independently being lower alkyl or as defined for the alkyl group above,

a 3 to 7 membered, saturated or unsaturated, heterocyclic ring unsubstituted or substituted with 1 or 2 substituent(s) each independently being lower alkyl or as defined for the alkyl group above,

a substituted or unsubstituted alkyl or alkenyl group as defined above incorporating as a group member a substituted or unsubstituted carbocyclic ring or a heterocyclic ring as defined above,

hydroxy, lower alkoxy, aryloxy, aryl lower alkoxy, amino, amino lower alkyl, lower alkyl amino, aryl amino or aryl lower alkyl amino, wherein the said alkyl, aryl or amino subgroups are unsubstituted or substituted with 1 to 3 substituent(s) each independently being lower alkyl or as defined for the alkyl group above;

R₂ is

a straight or branched alkyl chain having 1 to 5 carbon atoms unsubstituted or substituted with 1 or 2 substituent(s) each independently being hydroxy, oxo, lower alkoxy, amino, lower alkyl amino, halogen, carboxyl or lower acyl;

R₃ is:

H, cyano or COR⁴, wherein R⁴ is H, lower alkyl, cycloalkyl, cycloalkenyl, heterocycle or aryl, wherein the said lower alkyl is unsubstituted or substituted with 1 or 2 substituent(s) each independently being hydroxy, oxo, halogen, lower alkoxy, aryl, aryloxy, aryl lower alkoxy, cycloalkyl or heterocycle; or

wherein

R₁ is

a straight alkyl chain having 1 to 3 carbon atoms unsubstituted or substituted with 1 or 2 substituent(s) each independently being aryl, aryloxy, aryl lower alkoxy, lower alkyl amino, aryl amino, aryl lower alkyl amino, cycloalkyl or heterocycle,

a 3 to 7 membered, saturated or unsaturated, unsubstituted heterocyclic ring, lower alkoxy, lower alkyl amino, aryl amino or aryl lower alkyl amino;

R₂ is a straight or branched unsubstituted alkyl chain having 1 to 4 carbon atoms;

R₃ is:

H, cyano or COR⁴, wherein R⁴ is H or lower alkyl, wherein the said lower alkyl is unsubstituted or substituted with hydroxy.

Another possible subgroup of the compound of formula (I) is a compound wherein

X is C;

the dotted line represents a double bond;

R₁ is:

a straight or branched alkyl chain having 1 to 10 carbon atoms unsubstituted or substituted with 1 to 3 substituent(s) each independently being COOR⁴, COR⁴, CR⁴(OR⁵)₂, COCH₂OR⁶, cyano, hydroxy, oxo, halogen, lower alkoxy, aryl, aryloxy, aryl lower alkoxy, nitro, amino, lower alkyl amino, aryl amino, aryl lower alkyl amino, cycloalkyl or heterocycle, wherein R⁴ is H, lower alkyl, lower alkenyl, cycloalkyl, cycloalkenyl, heterocycle, aryl or aralkyl, R⁵ is lower alkyl, lower alkenyl, cycloalkyl, cycloalkenyl, aryl or aralkyl and R⁶ is H, lower alkyl, lower acyl or halogen, a straight or branched alkenyl chain having 2 to 10 carbon atoms unsubstituted or substituted with 1 to 3 substituent(s) as defined for the alkyl group above,

a 3 to 7 membered, saturated or unsaturated, carbocyclic ring unsubstituted or substituted with 1 to 3 substituent(s) each independently being lower alkyl or as defined for the alkyl group above,

a 3 to 7 membered, saturated or unsaturated, heterocyclic ring unsubstituted or substituted with 1 to 3 substituent(s) each independently being lower alkyl or as defined for the alkyl group above,

a substituted or unsubstituted alkyl or alkenyl group as defined above incorporating as a group member a substituted or unsubstituted carbocyclic ring or a heterocyclic ring as defined above,

hydroxy, lower alkoxy, aryloxy, aryl lower alkoxy, amino, amino lower alkyl, lower alkyl amino, aryl amino or aryl lower alkyl amino, wherein the said alkyl, aryl or amino subgroups are unsubstituted or substituted with 1 to 3 substituent(s) each independently being lower alkyl or as defined for the alkyl group above;

R₂ is H;

R₃ is:

H, cyano, hydroxy, oxo, halogen, lower alkyl, lower alkoxy, aryl, aryloxy, aryl lower alkoxy, amino, lower alkyl amino, aryl amino, aryl lower alkyl amino, cycloalkyl or heterocycle, wherein the said alkyl subgroups are unsubstituted or substituted with 1 to 3 substituent(s) as defined for the alkyl group, in the meaning of R₁, above,

or R₃ is COOR⁴, COR⁴, CR⁴(OR⁵)₂ or COCH₂OR⁶, wherein R⁴ is H, lower alkyl, lower alkenyl, cycloalkyl, cycloalkenyl, heterocycle, aryl, amino, lower alkyl amino, aryl amino or lower alkyl amino, wherein the said lower alkyl is unsubstituted or substituted with 1 or 2 substituent(s) each independently being cyano, hydroxy, oxo, halogen, lower alkoxy, aryl, aryloxy, aryl lower alkoxy, amino, lower alkyl amino, aryl amino, aryl lower alkyl amino, cycloalkyl or heterocycle, R⁵ is lower alkyl, lower alkenyl, cycloalkyl, cycloalkenyl, aryl or aralkyl and R⁶ is lower acyl or halogen, or a pharmaceutically acceptable salt or ester thereof; for example

wherein

R₁ is

a straight or branched alkyl chain having 1 to 5 carbon atoms unsubstituted or substituted with 1 or 2 substituent(s) each independently being hydroxy, halogen, lower alkoxy, aryl, aryloxy, aryl lower alkoxy, amino, lower alkyl amino, aryl amino, aryl lower alkyl amino, cycloalkyl or heterocycle,

a 3 to 7 membered, saturated or unsaturated, carbocyclic ring unsubstituted or substituted with 1 or 2 substituent(s) each independently being lower alkyl or as defined for the alkyl group above,

a 3 to 7 membered, saturated or unsaturated, heterocyclic ring unsubstituted or substituted with 1 or 2 substituent(s) each independently being lower alkyl or as defined for the alkyl group above,

a substituted or unsubstituted alkyl or alkenyl group as defined above incorporating as a group member a substituted or unsubstituted carbocyclic ring or a heterocyclic ring as defined above,

hydroxy, lower alkoxy, aryloxy, aryl lower alkoxy, amino, amino lower alkyl, lower alkyl amino, aryl amino or aryl lower alkyl amino, wherein the said alkyl, aryl or amino subgroups are unsubstituted or substituted with 1 to 3 substituent(s) each independently being lower alkyl or as defined for the alkyl group above;

R₃ is:

H, cyano or COR⁴, wherein R⁴ is H, lower alkyl, cycloalkyl, cycloalkenyl, heterocycle or aryl, wherein the said lower alkyl is unsubstituted or substituted with 1 or 2 substituent(s) each independently being hydroxy, oxo, halogen, lower alkoxy, aryl, aryloxy, aryl lower alkoxy, cycloalkyl or heterocycle; or

wherein

R₁ is

a straight or branched alkyl chain having 1 to 3 carbon atoms unsubstituted or substituted with 1 or 2 substituent(s) each independently being, aryl, aryloxy, aryl lower alkoxy, lower alkyl amino, aryl amino, aryl lower alkyl amino, cycloalkyl or heterocycle,

a 3 to 7 membered, saturated or unsaturated, unsubstituted heterocyclic ring, lower alkoxy, amino lower alkyl, lower alkyl amino, aryl amino or aryl lower alkyl amino, wherein the amino subgroups are unsubstituted or substituted with lower alkyl;

R₃ is:

H, cyano or COR⁴, wherein R⁴ is H or lower alkyl, wherein the said lower alkyl is unsubstituted or substituted with hydroxy.

The various substituents and groups used in this application are defined as follows.

“Lower alkyl” means a straight or branched saturated hydrogen carbon chain having 1 to 7, possibly 1 to 5 carbon atom(s). Representative examples include, but are not limited to, methyl, ethyl, propyl, isopropyl, butyl, sec-butyl, tert-butyl, pentyl, and the like.

“Lower alkenyl” means a straight or branched unsaturated hydrogen carbon chain having 2 to 7, possibly 2 to 5 carbon atoms, and containing (a) double bond(s). Representative examples include, but are not limited to, ethenyl, propenyl, butenyl, pentenyl, and the like.

“Lower alkynyl” means a straight or branched unsaturated hydrogen carbon chain having 2 to 7, possibly 2 to 5 carbon atoms, and containing (a) triple bond(s). Representative examples include, but are not limited to, ethynyl, propynyl, butynyl, pentynyl, and the like.

“Lower alkoxy” as such or in the group “aryl lower alkoxy”, is an alkoxy group having 1 to 7, possibly 1 to 5 carbon atom(s). Representative examples include, but are not limited to, methoxy, ethoxy, propoxy, isopropoxy, butoxy, sec-butoxy, tert-butoxy and pentoxy, phenyl methoxy, phenyl ethoxy, and the like.

“Lower alkyl amino” is an alkyl or dialkyl amino having 1 to 7 carbon atom(s) in the alkyl group(s). Representative examples include, but are not limited to, methyl amino, ethyl amino, propyl amino, isopropyl amino, butyl amino, pentyl amino, dimethyl amino, diethyl amino, N-ethyl-N-methyl amino, and the like.

“Lower acyl” is an acyl group having 2 to 7 carbon atoms. Representative examples include, but are not limited to, acetyl, propanoyl, isopropanoyl, butanoyl, sec-butanoyl, tert-butanoyl, pentanoyl, and the like.

A “cycloalkyl”, a “cycloalkenyl group” or a “carbocyclic ring” is a saturated or unsaturated cyclic hydrocarbon group containing 3 to 7, possibly 5 to 7 carbon atom(s). Representative examples include, but are not limited to, cyclopropyl, cyclobutyl, cyclopentyl, cyclopentenyl, cyclohexyl, phenyl, and the like.

A “heterocyclic ring” or a “heterocycle” group is a saturated or unsaturated 3 to 7, possibly 5 to 7 membered heterocyclic ring containing 1 to 3 heteroatom(s) selected from a nitrogen atom, an oxygen atom and/or sulphur atom. Representative examples include, but are not limited to, pyrrole, pyridine, pyrimidine, azepine, furan, pyran, oxepine, thiophene, thiopyran, thiepine, thiazole, imidazole, tetrazole, or their corresponding hydrated or partially hydrated derivatives, and the like.

“Aryl” as such or as a part of an “aralkyl”, especially an “aryl lower alkyl” group, or as a part of an “aryloxy” or “aryl amino” is an aromatic group with 6 to 12 carbon atoms, and is possibly a monocyclic aryl group, such as a phenyl group.

“Halogen atom” means chlorine, bromine, fluorine or iodine.

In general, the compounds of formula (I) can be synthesized starting from compounds 1a and 1b and compounds of the general structure 2 according to Schemes 1 and 2.

The compounds 1a and 1b are synthesized according to Nöteberg, D. et al. (J. Med. Chem. 2000, 43, 1705-1713).

Compounds of structure 2, with varying R2 groups and with or without varying protecting groups PG, are synthesized according to known synthesis methods described in the literature by for example Beausoleil, E. et al. (J. Org. Chem 1996, 61, 9447-9454), Collado, I. et al. (J. Org. Chem. 1995, 60, 5011-5015), Gershon, H. et al. (J. Org. Chem. 1961, 26, 2347-2350), Ho, T. L. et al. (J. Org. Chem 1986, 51, 2405-2408), Ibrahim, H. H. et al. (J. Org. Chem. 1993, 58, 6438-6441), Overberger, C. G. et al. (Macromolecules 1972, 5, 368-372), Pyne, S. G. et al. (Tetrahedron 1995, 51, 5157-5168), Sanno, Y. et al. (Yakugaku Zasshi 1958, 78, 1113-1118), Van der Werf, A. et al. (Tetrahedron Lett. 1991, 32, 3727-3730), Wei, L. et al. (Org. Lett. 2000, 2, 2595-2598), and Wistrand, L.-G. et al. (Tetrahedron 1991, 47, 573-582).

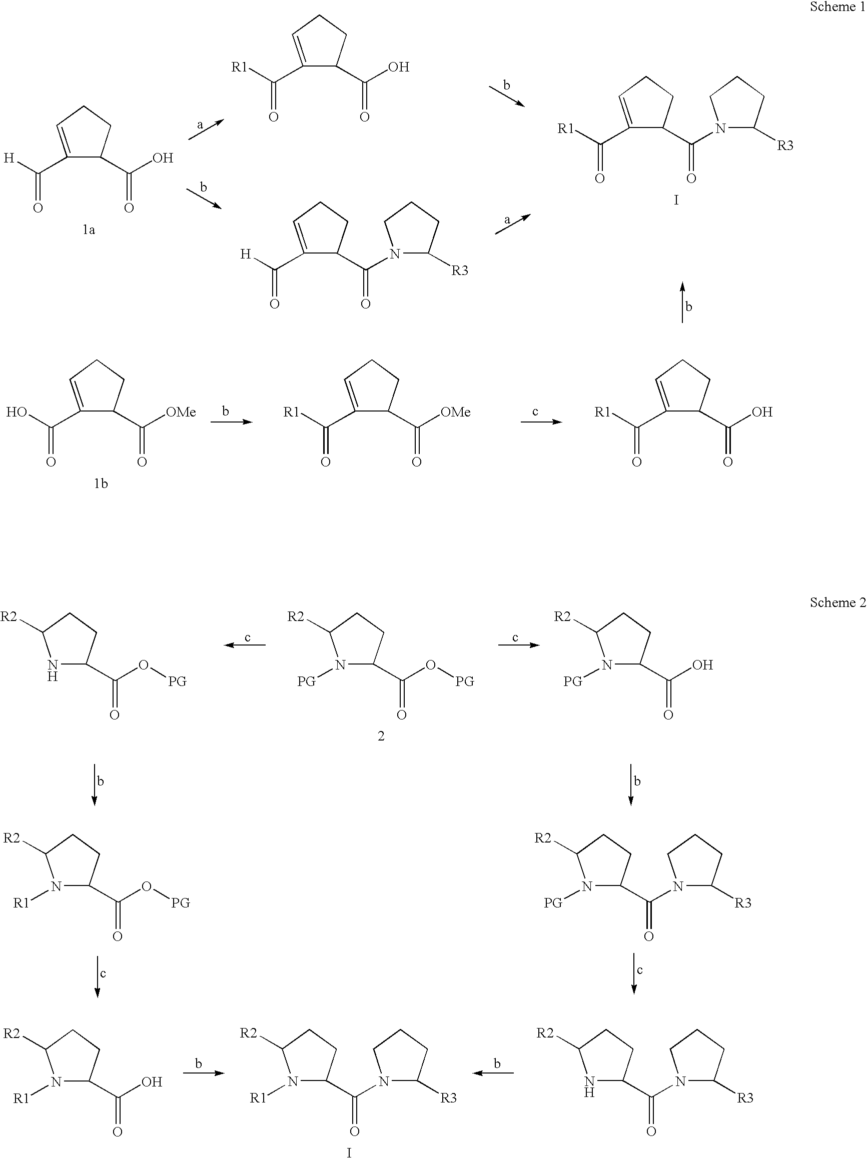

The reactions in Schemes 1 and 2 can be of the following types: a) formation of ketones from aldehydes and organometal reagents such as Grignard reagents, b) formation of amides from carboxylic acids and amines, and c) deprotection of protective groups such as esters and carbamates. All of these reaction are well known in the field of organic chemistry.

For the formation of a salt with the compounds of the formula (I) any suitable, pharmaceutically acceptable acid or base can be used, such as hydrochloric, hydrobromic, sulphuric, phosphoric or nitric acid, or an organic acid, such as acetic acid, propionic, succinic, glycolic, lactic, maleic, malonic, tartaric, citric, fumaric, methanesulfonic, p-toluene sulfonic and ascorbic acid, as well as salts with amino acids, such as aspartic and glutamic acid. Suitable inorganic bases are, for example, the alkali, earth alkaline metal or ammonium hydroxides and carbonates, as well as organic bases, such as organic amines, for example trialkyl amines, pyridine etc.

It has been found that the presence of the substituent R₂ in compounds, wherein X is N and the dotted line in the formula (I) represents a single bond, and the presence of the double bond represented by the dotted line in the formula (I) in compounds, wherein X is C, result in increased inhibitory activity.

The novel compounds according to the invention may be used to treat any condition, which responds to a treatment with a prolyl oligopeptidase inhibitor. The compound according to the invention can be administered for example orally, parenterally, topically or rectally by means of any pharmaceutical formulation useful for said administration, and containing the said compound in pharmaceutically acceptable and effective amounts together with pharmaceutically acceptable carriers, adjuvants or vehicles known in the art. The manufacture of such pharmaceutical formulations is well known in the art.

Thus the pharmaceutical composition may be in a dosage form suitable for oral use, such as tablets, capsules, liquid dosage forms, e.g. as suspensions, emulsions, syrups etc. All such formulations are made using per se known formulation techniques and carriers, adjuvants and additives. The compounds according to the invention may also be administered parenterally, e.g. for infusion and injection, for example using aqueous or oily suspensions, emulsions, or dispersions containing the active agent in combination with conventional pharmaceutically acceptable excipients. Formulations for rectal use are e.g. suppositories containing the active agent in combination with carrier substances suitable for rectal use.

The therapeutic dose to be given to a patient in need of treatment will vary depending on the body weight and age of the patient, the particular condition being treated, as well as the manner of administration, and are easily determined by a person skilled in the art. Typically a dosage form for oral use containing 0.01 mg to 5 g, typically 0.1 mg to 500 mg of active agent to be administered 1 to 3 times daily, would be suitable for most purposes.

The following examples illustrate the invention without limiting the same in any way.

General Synthesis Procedures

Positive ion mass spectra were acquired with ESI-MS, using a Finnegan MAT LCQ quadropole ion trap mass spectrometer equipped with an ESI source. Decoupled ¹³C NMR spectra were recorded on a Bruker Avance 500 spectrometer (125.8 MHz for ¹³C) or a Bruker AM 400 spectrometer (100.6 MHz for ¹³C), CDCl₃ was used as solvent and chemical shifts are expressed in ppm relative to tetramethylsilane as internal standard. Combustion analysis for CHN were measured on an EA1110 ThermoQuest CE Instruments elemental analysator. All chemicals and solvents were of commercial quality and were purified if necessary following standard procedures. Some intermediate products and all end products were purified by flash chromatography (30-60 μm Silica gel for flash, J. T. Baker) with a suitable eluent.

### Procedure A: General Procedure for Synthesis of 2-(1-hydroxy-alkyl)-cyclopent-2-ene-carboxylic acids

A solution of 2-formyl-cyclopent-2-ene-carboxylic acid (1.0 mmol) in anhydrous diethyl ether was added to the alkyl magnesium bromide (prepared from the corresponding alkyl bromide (2-4 mmol) and magnesium (2-4 mmol) in anhydrous diethyl ether using a crystal of iodine as the initiator) at rt. After 2 h the reaction mixture was poured into cold saturated NH₄Cl. The solution was made acidic with hydrochloric acid and the product was extracted with dichloromethane. The dichloromethane layer was dried and evaporated.

### Procedure B: General Procedure for Synthesis of 2-acyl-cyclopent-2-ene-carboxylic Acids

Dimethyl sulfoxide (2-3 mmol) was added to a solution of oxalyl chloride (1.0-1.5 mmol) in dichloromethane (4 ml) at −80° C. After 15 min a solution of 2-(1-hydroxy-alkyl)-cyclopent-2-ene-carboxylic acid (1.0 mmol) in dichloromethane (2 ml) was added. The reaction mixture was allowed to react for 1 h at −80° C., where after triethyl amine (4-6 mmol) was added. The reaction mixture was stirred further 5 min at −80° C. before it was allowed to warm to rt. The organic phase was extracted with 5% NaOH. The aqueous phase was made acidic with hydrochloric acid and the product was extracted with dichloromethane. The dichloromethane phase was dried and evaporated.

### Procedure C: General Procedure for Coupling an Amine to a Carboxylic Acid With Pivaloyl Chloride

Pivaloyl chloride (1.0 mmol) was added to a solution of the carboxylic acid (1.0 mmol) and triethyl amine (1.1 mmol) in dichloromethane at 0° C. After 1 h triethyl amine (1.1 mml, or if the amine is in the form of a HCl or trifluoroacetic acid salt then 3.3 mmol) and the amine (1.0-1.1 mmol) was added, where after the reaction mixture was allowed to react 3-20 h at rt. The dichloromethane solution was washed with 30% citric acid, saturated NaCl and saturated NaHCO₃. The dichloromethane phase was dried and evaporated.

### Procedure D: Procedure for Hydrolyzing a Methyl or Ethyl Ester Group

Lithium hydroxide (1.5-6.0 mmol) and carboxylic acid ester (1.0 mmol) were dissolved in a small volume of water-methanol. After the reaction was complete the solvent methanol was evaporated and water was added. The aqueous phase was washed with dichloromethane. The aqueous phase was then made acidic with hydrochloric acid and the product was extracted with dichloromethane. The dichloromethane phase was dried and evaporated.

### Procedure E: Deprotecting a Boc Protected Amine

The Boc protected amine (1.0 mmol) was dissolved in dichloromethane (5-10 ml) and trifluoroacetic acid (2-4 ml) was added at 0° C. The reaction was stirred at 0° C. for 2 h. The solvent was evaporated, yielding the trifluoroacetic acid salt of the amine.

### Procedure F: Hydrolysis of an O-acetyl Group

K₂CO₃ (1.1 mmol) was added to a solution of O-acetyl compound (1.0 mmol) in water-methanol (6 ml) at 0° C. The reaction was stirred 10 min at 0° C. and then 50 min at rt. The solvent methanol was evaporated. Dichloromethane and saturated NaCl were added and the phases were separated. The dichloromethane phase was washed once with saturated NaCl. The dichloromethane phase was dried and evaporated.

### Procedure G: Converting a Carboxylic acid to a Carboxylic Acid Amide

Ethyl chloroformate (1.0 mmol) was added to a solution of the carboxylic acid (1.0 mmol) and triethyl amine (1.0 mmol) in anhydrous tetrahydrofuran at −10° C. After 20 min 25% NH₃ (0.068 ml) was added at −10° C. The reaction mixture was stirred at rt overnight. The solvent was evaporated and the residue was dissolved in dichloromethane. The dichloromethane phase was washed with saturated NaHCO₃. The dichloromethane phase was then dried and evaporated.

### Procedure H: Converting a Carboxylic Acid Amide to a Cyano Group

Trifluoroacetic anhydride (1.5 mmol) was added to a solution of carboxylic acid amide (1.0 mmol) and triethyl amine (3 mmol) in anhydrous tetrahydrofuran. After 2-3 h water (10 ml) was added and the solvent was evaporated. The residue was dissolved in dichloromethane. The dichloromethane solution was washed with 30% citric acid, saturated NaCl and saturated NaHCO₃. The dichloromethane phase was then dried and evaporated.

### PREPARATION OF STARTING MATERIALS

### L-Proline Methyl Ester HCl Salt

Thionyl chloride (16 ml, 220 mmol) was added to a solution of L-proline (10 g, 87 mmol) in methanol (200 ml) at 0° C. The reaction mixture was refluxed for 1 h. The solvent was evaporated, yield 14 g (86 mmol).

### Boc-2(S)-(acetoxyacetyl)pyrrolidine

Ethyl chloroformate (3.14 ml, 33 mmol) was added to a solution of Boc-L-proline (6.46 g, 30 mmol) and triethyl amine (4.60 ml, 33 mmol) in anhydrous tetrahydrofuran (100 ml) at −20° C. The reaction mixture was stirred at −20° C. for 30 min. Then a diethyl ether solution of diazomethane (prepared according to Aldrich Technical Bulletin AL-180 from N-methyl-N-nitroso-4-toluenesulfonamide (6.4 g, 30 mmol)) was added to the reaction mixture at −20° C. The reaction mixture was stirred at −20° C. for 1 h, where after the reaction mixture was left without stirring at −20° C. overnight. Toluene (120 ml) was added, and the organic phase was washed with saturated NaHCO₃ and water. The organic phase was dried and evaporated. The residue was dissolved acetic acid (30 ml) and the solution was stirred at 100° C. for 10 min. The reaction mixture was evaporated. The residue was dissolved in ethyl acetate and the solution was washed with saturated NaHCO₃ and water. The ethyl acetate phase was dried and evaporated. The product was purified by flash chromatography, yield 1.94 g (7.2 mmol).

### SYNTHESIS OF THE PRODUCT COMPOUNDS

### EXAMPLE 1

### 2-(Benzylcarbamoyl)-cyclopent-2-ene-carboxylic acid methyl ester

Dicyclohexylcarbodiimide (3.06 g, 14.8 mmol) was added to a solution of cyclopent-2-ene-1,2-dicarboxylic acid 1-methyl ester (1.68 g, 9.9 mmol), benzyl amine (1.62 ml, 14.8 mmol), hydroxybenzotriazole (2.27 g, 14.8 mmol) and triethyl amine (2.07 ml, 14.8 mmol) in acetonitrile at 0° C. After 30 min the reaction was allowed to warm to rt and it was left at rt overnight. The solvent was evaporated and the residue was dissolved in dichloromethane. The dichloromethane solution was washed with saturated NaHCO₃, saturated NaCl and 30% citric acid. The dichloromethane phase was dried and evaporated. Purification by flash chromatography, yield 2.58 g (9.9 mmol).

### 2-(Benzylcarbamoyl)-cyclopent-2-ene-carboxylic acid

The methyl ester group of 2-benzylcarbamoyl-cyclopent-2-ene-carboxylic acid methyl ester (2.58 g, 9.9 mmol) was hydrolyzed according to procedure D. Yield 2.19 g (8.9 mmol).

### 2-(Benzylcarbamoyl)-cyclopent-2-ene-carboxylic acid (L-proline methyl ester) amide

2-(Benzylcarbamoyl)-cyclopent-2-ene-carboxylic acid (2.19 g, 8.9 mmol) and proline methyl ester (1.48 g, 8.9 mmol) were coupled according to procedure C. Purification by flash chromatography, yield 2.64 g (7.4 mmol).

### 2-(Benzylcarbamoyl)-cyclopent-2-ene-carboxylic acid L-proline amide

The methyl ester group of 2-(benzylcarbamoyl)-cyclopent-2-ene-carboxylic acid (L-proline methyl ester) amide (2.64 g, 7.4 mmol) was hydrolyzed according to procedure D.

Yield 2.32 g (6.8 mmol).

### 2-(Benzylcarbamoyl)-cyclopent-2-ene-carboxylic acid L-prolylamide amide

Prepared according to procedure G using 2-(benzylcarbamoyl)-cyclopent-2-ene-carboxylic acid (2.32 g, 6.8 mmol) as the starting material. Purification by flash chromatography, yield 2.3 g (6.8 mmol).

### 2-(Benzylcarbamoyl)-cyclopent-2-ene-carboxylic acid 2(S)-cyanopyrrolidine amide

Prepared according to procedure H using 2-(benzylcarbamoyl)-cyclopent-2-ene-carboxylic acid Lprolylamide amide (2.3 g, 6.8 mmol). Purification and separation of diastereomers by flash chromatography, yield of one of the diastereomers 0.12 g, (0.37 mmol).

¹³C NMR: δ 25.22, 27.88, 30.00, 33.04, 43.43, 46.47, 46.76, 48.99, 118.73, 127.41, 127.64, 128.69, 137.80, 138.27, 139.45, 165.06, 173.96.

Anal. (C₁₉H₂₁N₃O₂.0.3 H₂O) calcd C: 69.41, H: 6.62, N: 12.78; found C: 69.51, H: 6.54, N: 12.58.

### EXAMPLE 2

### 2-Benzylcarbamoyl-cyclopent-2-ene-carboxylic acid 2(S)-(acetoxyacetyl)-pyrrolidine amide

2-Benzylcarbamoyl-cyclopent-2-ene-carboxylic acid (0.86 g, 3.5 mmol) and 2(S)-(acetoxyacetyl)pyrrolidine trifluoroacetic acid salt (prepared from Boc-2(S)-(acetoxyacetyl)pyrrolidine (0.95 g, 3.5 mmol) according to procedure E) were coupled according to procedure C. Purification by flash chromatography, yield 0.82 g (2.1 mmol).

### 2-Benzylcarbamoyl-cyclopent-2-ene-carboxylic acid 2(S)-(hydroxyacetyl)-pyrrolidine amide

The acetyl group of 2-benzylcarbamoyl-cyclopent-2-ene-carboxylic acid 2(S)-(acetoxyacetyl)-pyrrolidine amide (0.82 g, 2.1 mmol) was hydrolyzed according to procedure F. Purification and separation of diastereomers by flash chromatography, yield of the more active diastereomer 0.21 g (0.58 mmol).

¹³C NMR: δ 25.15, 27.55, 28.51, 32.94, 43.47, 47.80, 49.00, 61.20, 67.06, 127.40, 127.64, 128.66, 138.24, 138.36, 139.11, 165.80, 174.21, 209.28.

ESI-MS: m/z 357 (M+H)⁺.

Anal. (C₂₀H₂₄N₂O₄.0.1 H₂O) calcd C: 67.06, H: 6.81, N: 7.82; found C: 66.98, H: 6.86, N: 7.62.

### EXAMPLE 3

### 2-Benzylcarbamoyl-cyclopent-2-ene-carboxylic acid pyrrolidine amide

2-Benzylcarbamoyl-cyclopent-2-ene-carboxylic acid (0.46 g, 1.9 mmol) and pyrrolidine (0.16 ml, 1.9 mmol) were coupled according to procedure C. Purification by flash chromatography, yield of the racemic product 0.39 g (1.3 mmol).

¹³C NMR: δ 24.36, 26.13, 28.12, 32.75, 43.36, 45.93, 46.90, 49.50, 127.21, 127.64, 128.57, 137.55, 138.60, 140.05, 165.61, 173.22.

ESI-MS: m/z 299 (M+H)⁺.

Anal. (Cl₈H₂₂N₂O₂.0.2 H₂O) calcd C: 71.59, H: 7.48, N: 9.28; found C: 71.43, H: 7.55, N: 9.19.

### EXAMPLE 4

### 2-(1-Hydroxy-2-phenyl-ethyl)-cyclopent-2-ene-carboxylic acid

Prepared according to procedure A using 2-formyl-cyclopent-2-ene-carboxylic acid (2.1 g, 15.0 mmol) and benzyl bromide (7.2 ml, 60 mmol) as the starting materials. Purification by flash chromatography, yield 0.80 g (3.5 mmol).

### 2-Benzylcarbonyl-cyclopent-2-ene-carboxylic acid

2-(1 -Hydroxy-2-phenyl-ethyl)-cyclopent-2-ene-carboxylic acid (0.26 g, 1.1 mmol) was oxidized according to procedure B. Purification by flash chromatography, yield 0.074 g (0.32 mmol).

### 2-Benzylcarbonyl-cyclopent-2-ene-carboxylic acid pyrrolidine amide

2-Benzoyl-cyclopent-2-ene-carboxylic acid (0.14 g, 0.61 mmol) and pyrrolidine (0.051 ml, 0.67 mmol) were coupled according to procedure C. Purification by flash chromatography, yield of the racemic product 0.12 g (0.42 mmol).

¹³C-NMR: δ 24.43, 26.11, 28.15, 33.79, 45.67, 45.84, 46.89, 47.92, 126.72, 128.52, 129.50, 134.88, 145.20, 146.72, 172.83, 195.46.

ESI-MS: m/z 284 (M+H)⁺.

Anal. (C₁₈H₂₁NO₂) calcd C: 76.30, H: 7.47, N: 4.94; found: C: 76.17, H: 7.69, N: 4.94.

### EXAMPLE 5

### 2-(1-Hydroxy-4-phenyl-butyl)-cyclopent-2-ene-carboxylic acid

Prepared according to procedure A using 2-formyl-cyclopent-2-ene-carboxylic acid (2.1 g, 15 mmol) and 1-brom-3-phenylpropane (4.8 g, 31.5 mmol) as the starting materials. Purification by flash chromatography, yield 1.31 g (5.0 mmol).

### 2-(4-Phenylbutanoyl)-cyclopent-2-ene-carboxylic acid

2-(1-Hydroxy-4-phenyl-butyl)-cyclopent-2-ene-carboxylic acid (1.31 g, 5.0 mmol) was oxidized according to procedure B. Purification by flash chromatography, yield 0.39 g (1.5 mmol).

### 2-(4-Phenylbutanoyl)-cyclopent-2-ene-carboxylic acid (L-proline methyl ester) amide

2-(4-Phenylbutanoyl)-cyclopent-2-ene-carboxylic acid (0.58 g, 2.3 mmol) and proline methyl ester (0.37 g, 2.3 mmol) were coupled according to procedure C. Yield 0.64 g (1.7 mmol).

### 2-(4-Phenylbutanoyl)-cyclopent-2-ene-carboxylic acid L-proline amide

The methyl ester group of 2-(4-phenylbutanoyl)-cyclopent-2-ene-carboxylic acid (L-proline methyl ester) amide (0.64 g, 1.7 mmol) was hydrolyzed according to procedure D.

Yield 0.58 g (1.6 mmol).

### 2-(4-Phenylbutanoyl)-cyclopent-2-ene-carboxylic acid L-prolylamide amide

Prepared according to procedure G using 2-(4-phenylbutanoyl)-cyclopent-2-ene-carboxylic acid L-proline amide (0.58 g, 1.6 mmol) as starting material. Purification by flash chromatography, yield 0.50 g (1.4 mmol).

### 2-(4-Phenylbutanoyl)-cyclopent-2-ene-carboxylic acid 2(S)-cyanopyrrolidine amide

Prepared according to procedure H using 2-(4-phenylbutanoyl)-cyclopent-2-ene-carboxylic acid L-prolylamide amide (0.50 g, 1.4 mmol). Purification and sepapration of diastereomers by flash chromatography, yield of the more active diastereomer 190 mg (0.56 mmol).

¹³C NMR: δ 24.74, 25.20, 27.41, 29.52, 33.16, 34.62, 37.33, 45.97, 46.29, 47.00, 118.31, 125.41, 127.84, 127.98, 141.10, 144.10, 145.86, 173.20, 197.84.

ESI-MS: m/z 337.0 (M+H)⁺.

Anal. (C₂₁H₂₄N₂O₂.0.1 H₂O) calcd C: 74.57, H: 7.21, N: 8.28; found C: 74.28, H: 7.53, N: 7.93.

### EXAMPLE 6

### 2-(4-Phenylbutanoyl)-cyclopent-2-ene-carboxylic acid pyrrolidine amide

2-(4-Phenylbutanoyl)-cyclopent-2-ene-carboxylic acid (0.23 g, 0.89 mmol) and pyrrolidine (0.074 ml, 0.89 mmol) were coupled according to procedure C. Purification by flash chromatography, yield of the racemic product 0.21 g (0.69 mmol).

¹³C NMR: δ 24.45, 25.68, 26.15, 28.07, 33.56, 35.19, 37.99, 45.82, 46.89, 47.84, 125.84, 128.31, 128.53, 141.80, 145.27, 145.39, 172.92, 198.28.

ESI-MS: m/z 312 (M+H)⁺.

Anal. (C₂₀H₂₅NO₂) calcd C: 77.14, H: 8.09, N: 4.50; found C: 77.09, H: 8.30, N: 4.38.

### EXAMPLE 7

### (2S)5-Oxo-2-[N-(benzyloxycarbonyl)-amino]hexanoic acid methyl ester

(2S)-5-Oxo-2-[N-(benzyloxycarbonyl)-amino]hexanoic acid (3.45 g, 12.3 mmol) (prepared according to Ho, T. L. et al. (J. Org. Chem. 1986, 51, 2405-2408)) was methylated with a small excess of diazomethane (prepared according to Aldrich Technical Bulletin AL-180) in anhydrous tetrahydrofuran at 0° C. The reaction mixture was left at 4° C. overnight. The solvent was evaporated and the residue was dissolved in diethyl ether. The diethyl ether phase was washed with water and saturated NaHCO₃. The diethylether phase was dried and evaporated. Purification by flash chromatography, yield 1.5 g (5.1 mmol).

### Boc-5(R)-methyl-L-proline methyl ester

Prepared by reacting (2S)-5-oxo-2-[N-(benzyloxycarbonyl)-amino]hexanoic acid methyl ester 1.5 g (5.1 mmol) and di-tert-butyl-dicarbonat (3.1 g, 14.0 mmol) with 10% Pd/C (0.28 g) in methanol under 4 atm pressure of H₂ overnight. The solution was filtered through Celite and evaporated. Purification by flash chromatography, yield 0.90 g (3.7 mmol).

### 4-Phenylbutanoyl-5(R)-methyl-L-proline ethyl ester

4-Phenylbutanoylchloride (prepared from 4-phenylbutanoic acid (0.73 g, 4.4 mmol) and thionyl chloride (0.64 ml, 8.9 mmol)) was added to a solution of the 5(R)-methyl-L-proline ethyl ester trifluroacetic acid salt (prepared from Boc-5(R)-methyl-L-proline ethyl ester (0.90 g, 3.7 mmol) according to procedure E) and triethyl amine (2.1 ml, 15.0 mmol) in dichloromethane at 0° C., where after it was stirred at rt for 3 h. The dichloromethane phase was washed with 30% citric acid, saturated NaCl and saturated NaHCO₃. The dichloromethane phase was dried and evaporated. Purification by flash chromatography, yield 0.74 g (2.6 mmol).

### 4-Phenylbutanoyl-5(R)-methyl-L-proline

The ethyl ester group of 4-phenylbutanoyl-5(R)-methyl-L-proline ethyl ester (0.74 g, 2.6 mmol) was hydrolyzed according to procedure D. Yield 0.67 g (2.4 mmol).

### 4-Phenylbutanoyl-5(R)-methyl-L-prolyl-pyrrolidine

4-Phenylbutanoyl-5(R)-methyl-L-proline (0.67 g, 2.4 mmol) and pyrrolidine (0.22 ml, 2.7 mmol) were coupled according to procedure C. Purification by flash chromatography, yield 0.53 g (1.6 mmol).

¹³C NMR: δ 20.51, 24.16, 26.21, 26.22, 26.99, 32.85, 32.89, 35.21, 46.02, 46.35, 54.28, 58.87, 125.80, 128.27, 128.52, 141.75, 170.69, 171.03.

Anal. (C₂₀H₂₈N₂O₂.0.3 H₂O) calcd C: 71.95, H: 8.63, N: 8.39; found C: 72.14, H: 8.76, N: 8.34.

### EXAMPLE 8

### 4-Phenylbutanoyl-5(R)-methyl-L-prolyl-2(S)-(acetoxyacetyl)-pyrrolidine

4-Phenylbutanoyl-5(R)-methyl-L-proline (0.23 g, 0.84 mmol) and 2(S)-(acetoxyacetyl)-pyrrolidine trifluoroacetic acid salt (prepared from Boc-2(S)-(acetoxyacetyl)-pyrrolidine (0.23 g, 0.84 mmol) according to procedure E) were coupled according to procedure C. Purification by flash chromatography, yield 0.23 g (0.54 mmol).

### 4-Phenylbutanoyl -5(R)-methyl-L-prolyl-2(S)-(hydroxyacetyl)-pyrrolidine

Prepared according to procedure F using 4-phenylbutanoyl-5(R)-methyl-L-prolyl-2(S)-(acetoxyacetyl)-pyrrolidine (0.23 g, 0.54 mmol) as starting material. Purification by flash chromatography, yield 0.11 g (0.29 mmol).

¹³C NMR: δ 20.65, 25.34, 26.23, 26.82, 28.25, 32.84, 32.90, 35.23, 47.19, 54.30, 58.56, 61.27, 66.96, 125.88, 128.32, 128.50, 141.66, 171.21, 171.33, 209.05.

ESI-MS: m/z 387 (M+H)⁺.

Anal. (C₂₂H₃₀N₂O₄.0.5 H₂O) calcd C: 66.81, H: 7.90, N: 7.08; found C: 66.82, H: 76.83, N: 6.83.

### EXAMPLE 9

### Boc-5(R)-tert-butyl-L-proline methyl ester

Prepared according to Lubell, W. D. et al. (J. Org. Chem. 1996, 61, 9447-9454), with the small modification that the 9-(9-phenylfluorenyl) protecting group was replaced by the trityl protecting group in the synthesis procedure. The major diastereomer was isolated by flash chromatography.

### Boc-5(R)-tert-butyl-L-proline

The methyl ester group of Boc-5(R)-tert-butyl-L-proline methyl ester (1.14 g, 4.0 mmol) was hydrolyzed according to procedure D. Yield 0.88 g (3.2 mmol).

### Boc-5(R)-tert-butyl-L-prolyl-pyrrolidine

Boc-5(R)-tert-butyl-L-proline (0.88 g, 3.2 mmol) and pyrrolidine (0.27 ml, 3.2 mmol) were coupled according to procedure C. Purification by flash chromatography, yield 0.87 g (2.7 mmol).

¹³C NMR: δ 24.09, 26.35, 27.08, 27.59, 28.38, 28.85, 36.36, 45.96, 45.99, 61.00, 66.69, 79.60, 156.21, 171.15.

ESI-MS: m/z 325 (M+H)⁺.

Anal. (C₁₈H₃₂N₂O₃) calcd C: 66.63, H: 9.94, N: 8.63; found C: 66.28, H: 9.95, N: 8.57.

### EXAMPLE 10

### Acetyl-5(R)-tert-butyl-L-prolyl-pyrrolidine

Acetic anhydride (0.15 ml, 1.5 mmol) was added to a solution of the 5(R)-tert-butyl-L-prolyl-pyrrolidine trifluoroacetic acid salt (prepared from Boc-5(R)-tert-butyl-L-prolyl-pyrrolidine (0.25 g, 0.77 mmol) according to procedure E) and triethyl amine (0.40 ml, 3.1 mmol) in dichloromethane at 0° C. The reaction was stirred at rt for 3 h. The dichloromethane solution was washed with 30% citric acid, saturated NaCl and saturated NaHCO₃. The dichloromethane phase was dried and evaporated. Purification by flash chromatography, yield 0.17 g (0.65 mmol).

¹³C NMR: δ 22.74, 23.17, 23.94, 24.08, 26.25, 26.29, 26.42, 27.61, 27.95, 28.12, 29.65, 36.62, 36.64, 45.97, 45.98, 46.01, 46.31, 60.78, 61.81, 65.64, 68.18, 170.30, 170.46, 172.00, 172.02 (all except one carbon give double peaks).

ESI-MS: m/z 267 (M+H)⁺.

Anal. (C₁₅H₂₆N₂O₂) calcd C: 67.63, H: 9.84, N: 10.52; found C: 67.79, H: 10.16, N: 10.68.

### EXAMPLE 11

### 4-Phenylbutanoyl-5(R)-tert-butyl-L-prolyl-pyrrolidine

4-Phenylbutanoylchloride (prepared from 4-phenylbutanoic acid (0.39 g, 2.4 mmol) and thionyl chloride (0.21 ml, 2.9 mmol)) was added to a solution of the 5(R)-tert-butyl-L-prolyl-pyrrolidine trifluroacetic acid salt (prepared from Boc-5(R)-tert-butyl-L-prolyl-pyrrolidine (0.63 g, 1.9 mmol) according to procedure E) and triethyl amine (0.89 ml, 6.4 mmol) in dichloromethane at 0° C. The reaction mixture was stirred at rt for 3 h. The dichloromethane phase was washed with 30% citric acid, saturated NaCl and saturated NaHCO₃. The dichloromethane phase was dried and evaporated. Purification by flash chromatography, yield 0.61 g (1.6 mmol).

¹³C NMR: δ 23.90, 24.09, 25.92, 26.18, 26.34, 26.78, 27.41, 27.68, 27.93, 28.12, 29.60, 29.71, 33.07, 33.88, 35.12, 35.27, 36.44, 36.62, 45.76, 45.97, 46.00, 46.17, 60.82, 60.99, 65.72, 67.04, 125.74, 125.86, 128.25, 128.30, 128.51, 128.62, 141.75, 142.03, 170.34, 170.53, 173.99, 174.26.

ESI-MS: m/z 371 (M+H)⁺.

Anal. (C₂₃H₃₄N₂O₂.0.2 H₂O) calcd C: 73.84, H: 9.27, N: 7.49; found C: 73.91, H: 9.35, N: 7.17.

### EXAMPLE 12

### 4-Phenylbutanoyl-5(R)-tert-butyl-L-proline methyl ester

4-Phenylbutanoylchloride (prepared from 4-phenylbutanoic acid (0.76 g, 4.6 mmol) and thionyl chloride (0.50 ml, 6.9 mmol)) was added to a solution of the 5(R)-tert-butyl-L-proline methyl ester trifluroacetic acid salt (prepared from Boc-5(R)-tert-butyl-L-proline methyl ester (1.1 g, 3.8 mmol) according to procedure E) and triethyl amine (2.1 ml, 15.3 mmol) in dichloromethane at 0° C. The reaction was stirred 4 h in rt. The dichloromethane solution was washed with 30% citric acid, saturated NaCl and saturated NaHCO₃. The dichloromethane phase was dried and evaporated. Purification by flash chromatography, yield 0.73 g (2.2 mmol).

### 4-Phenylbutanoyl-5(R)-tert-butyl-L-proline

The methyl ester group of 4-phenylbutanoyl-5(R)-tert-butyl-L-proline methyl ester (0.68 g, 2.1 mmol) was hydrolyzed according to procedure D. Yield 0.58 g (1.8 mmol).

### 4-Phenylbutanoyl-5(R)-tert-butyl-L-prolyl-2(S)-(acetoxyacetyl)-pyrrolidine

4-Phenylbutanoyl-5(R)-tert-butyl-L-proline (0.58 g, 1.8 mmol) and 2(S)-(acetoxyacetyl)-pyrrolidine trifluoroacetic acid salt (prepared from Boc-2(S)-(acetoxyacetyl)-pyrrolidine (0.50 g, 1.8 mmol) according to procedure E) were coupled accoroding to procedure C. Purification by flash chromatography, yield 0.30 g (0.64 mmol).

### 4-Phenylbutanoyl-5(R)-tert-butyl-L-prolyl-2(S)-(hydroxyacetyl)-pyrrolidine

Prepared according to procedure F using 4-phenylbutanoyl-5(R)-tert-butyl-L-prolyl-2(S)-(acetoxyacetyl)-pyrrolidine (0.30 g, 0.64 mmol) as starting material. Purification by flash chromatography, yield 0.26 g (0.61 mmol).

¹³C NMR: δ 25.37, 25.42, 25.82, 26.06, 26.76, 27.15, 27.57, 27.82, 28.06, 28.07, 29.15, 29.43, 33.01, 33.79, 34.97, 35.24, 36.43, 36.53, 46.50, 46.79, 60.44, 60.63, 61.24, 61.30, 65.83, 66.90, 66.97, 67.08, 125.77, 125.91, 128.26, 128.33, 128.49, 128.65, 141.64, 141.97, 170.78, 171.01, 173.74, 174.39, 208.42, 209.31.

ESI-MS: m/z 429 (M+H)⁺.

Anal. (C₂₅H₃₆N₂O₄.0.1 H₂O) calcd C: 69.77, H: 8.48, N: 6.51; found C: 69.62, H: 8.48, N: 6.73.

### EXAMPLE 13

### Benzylcarbamoyl-5(R)-tert-butyl-L-prolyl-pyrrolidine

Benzylisocyanate (0.55 ml, 4.5 mmol) was added to a solution of the 5(R)-tert-butyl-L-proline methyl ester trifluroacetic acid salt (prepared from Boc-5(R)-tert-butyl-L-proline methyl ester (1.46 g, 4.5 mmol) according to procedure E) and triethyl amine (1.9 ml, 13.5 mmol) in dimethylformarnide at 0° C. The reaction was stirred 3 h in rt. The dimethylformamide solution was poured into ice-water and the product was extracted with dichloromethane. The dichloromethane phase was washed with 30% citric acid, saturated NaCl and saturated NaHCO₃. The dichloromethane phase was dried and evaporated. Purification by flash chromatography, yield 1.24 g (3.5 mmol).

¹³C NMR: δ 23.90, 26.34, 26.84, 27.54, 29.32, 36.46, 44.96, 46.16, 46.33, 62.56, 66.51, 127.07, 127.41, 128.54, 139.56, 160.29, 171.54.

Anal. (C₂₁H₃₁N₃O₂) calcd C: 70.55, H: 8.74, N: 11.75; found C: 70.72, H: 8.85, N: 12.08.

### EXAMPLE 14

### Boc-5(S)-methyl-L-proline ethyl ester

Prepared according to Collado, I. et al. (J. Org. Chem. 1995, 60, 5011-5015). Purification without separating the diastereomers by flash chromatography. This procedure yields the (2S,5S) diastereomer as the as the major product.

### 4-Phenylbutanoyl-5(S)-methyl-L-proline ethyl ester

4-Phenylbutanoylchloride (prepared from 4-phenylbutanoic acid (1.42 g, 8.6 mmol) and thionyl chloride (0.93 ml, 13.0 mmol)) was added to a solution of the 5(S)-methyl-L-proline ethyl ester trifluroacetic acid salt (prepared from Boc-5(S)-methyl-L-proline ethyl ester (1.85 g, 7.2 mmol) according to procedure E) and triethyl amine (4.0 ml, 28.7 mmol) in dichloromethane at 0° C. The reaction was stirred 3 h in rt. The dichloromethane phase was washed with 30% citric acid, saturated NaCl and saturated NaHCO₃. The dichloromethane phase was dried and evaporated. Purification by flash chromatography, yield 1.56 g (5.1 mmol).

### 4-Phenylbutanoyl-(S)-methyl-L-proline

The ethyl ester group of 4-phenylbutanoyl-5(S)-methyl-L-proline ethyl ester (1.54 g, 5.1 mmol) was hydrolyzed according to procedure D. Yield 1.36 g (4.9 mmol).

### 4-Phenylbutanoyl-5(S)-methyl-L-prolyl-pyrrolidine

4-Phenylbutanoyl-S(S)-methyl-L-proline (0.67 g, 2.4 mmol) and pyrrolidine (0.20 ml, 2.4 mmol) were coupled according to procedure C. Purification by flash chromatography, yield 0.64 g (2.0 mmol).

¹³C NMR: δ 21.72, 24.15, 26.25, 26.51, 26.54, 31.72, 32.99, 35.11, 45.87, 46.22, 53.72, 58.06, 125.76, 128.26, 128.64, 141.95, 170.53, 171.70.

Anal. (C₂₀H₂₈N₂₂.0.2 H₂O) calcd C: 72.34, H: 8.62, N: 8.44; found C: 72.08, H: 8.86, N: 8.55.

### EXAMPLE 15

### 4-Phenylbutanoyl-5(S)-methyl-L-prolyl-2(S)-(acetoxyacetyl)-pyrrolidine

Prepared according to procedure C using 4-phenylbutanoyl-5(S)-methyl-L-proline (0.69 g, 2.5 mmol) and 2(S)-(acetoxyacetyl)-pyrrolidine trifluoroacetic acid salt (prepared from Boc-2(S)-(acetoxyacetyl)-pyrrolidine (0.68 g, 2.5 mmol) according to procedure E). Purification by flash chromatography, yield 0.26 g (0.61 mmol).

### 4-Phenylbutanoyl -5(S)-methyl-L-prolyl-2(S)-(hydroxyacetyl)-pyrrolidine

Prepared according to procedure F using 4-phenylbutanoyl-5(S)-methyl-L-prolyl-2(S)-(acetoxyacetyl)-pyrrolidine (0.26 g, 0.61 mmol) as starting material. Purification by flash chromatography, yield 0.15 g (0.38 mmol).

¹³C NMR: δ 21.58, 25.34, 26.12, 26.44, 28.19, 31.60, 32.95, 35.14, 46.99, 53.81, 57.69, 60.94, 67.06, 125.83, 128.29, 128.55, 141.79, 171.01, 171.79, 209.19.

ESI-MS: m/z 387 (M+H)⁺.

Anal. (C₂₂H₃₀N₂O₄.0.4 H₂O) calcd C: 67.12, H: 7.89, N: 7.12; found C: 67.19, H: 7.88, N: 6.95.

### EXAMPLE 16

### Boc-5(S)-tert-butyl-L-proline ethyl ester

CuBr·Me₂S (4.11 g, 20 mmol) in anhydrous tetrahydrofuran (40 ml) was cooled to −80° C. and 1.5 M tert-butyllithium (13.3 ml, 20 mmol) was added. After 30 min BF₃.Et₂O (2.5 ml, 20 mmol) was added and after further 20 min a solution of Boc-5-methoxy-L-proline ethyl ester (1.28 g, 4.7 mmol) (prepared according to Collado, I. et al. (J. Org. Chem. 1995, 60, 5011-5015)) in anhydrous tetrahydrofuran (10 ml) was added. The reaction mixture was stirred for 15 min at −80° C., where after it was allowed to warm to room temperature during 3 h. A mixture of 25% NH₃ (12 ml) and saturated NH₄Cl (12 ml) was added and the reaction was stirred 1 h at room temperature. The tetrahydrofuran layer was separated and evaporated. The residue was dissolved in diethyl ether. The remaining aqueous layer was extracted with diethyl ether. Both diethyl ether layers were combined and washed with saturated NaHCO₃, dried and evaporated. Purification by flash chromatography without separation of diastereomers, yield 1.27 g (4.2 mmol). This procedure yields the (2S,5S)-diastereomer as the major product.

### Boc-5(S)-tert-butyl-L-proline

The ethyl ester group of Boc-5(S)-tert-butyl-L-proline ethyl ester (1.23 g, 4.1 mmol) was hydrolyzed according to procedure D with prolonged reaction time. Yield 0.62 g (2.3 mmol).

### Boc-5(S)-tert-butyl-L-prolyl-pyrrolidine

Boc-5(S)-tert-butyl-L-proline (0.62 g, 2.3 mmol) and pyrrolidine (0.19 ml, 2.3 mmol) were coupled according to procedure C. Purification by flash chromatography, yield 0.43 g (1.3 mmol).

¹³C NMR: δ 24.19, 25.03, 26.33, 27.52, 28.24, 29.66, 36.89, 45.91, 46.06, 60.18, 66.25, 79.01, 155.79, 172.02.

ESI-MS: m/z 325 (M+H)⁺.

Anal. (C₁₈H₃₂N₂O₃) calcd C: 66.63, H: 9.94, N: 8.63; found C: 66.77, H: 10.30, N: 8.75.

### EXAMPLE 17

### (±)-2-Formyl-cyclopent-2-enecarboxylic acid pyrrolidine amide

2-Formyl-cyclopent-2-enecarboxylic acid (0.50 g, 3.6 mmol) and pyrrolidine (0.30 ml, 3.6 mmol) were coupled according to procedure C. Purification by flash chromatography, yield 0.50 g (2.6 mmol).

### 2-(Hydroxy-pyridin-3-yl-methyl)-cyclopent-2-enecarboxylic acid pyrrolidine amide

To a solution of 3-iodopyridine (0.29 g, 1.4 mmol) in 10 ml of anhydrous THF was added 1 M solution of ethylmagnesium bromide in THF (1.7 ml, 1.7 mmol) at rt. After 30 min, (±)-2-formyl-cyclopent-2-enecarboxylic acid pyrrolidine amide (0.25 g, 1.3 mmol) in anhydrous THF was added and the mixture was stirred for 4 h. The reaction mixture was poured into cold saturated NH₄Cl and the solution was acidified with hydrochloric acid and washed with DCM. Purification by flash chromatography, yield 0.17 g (0.62 mmol).

### 2-Nicotinoyl-cyclopent-2-enecarboxylic acid pyrrolidine amide

2-(Hydroxy-pyridin-3-yl-methyl)-cyclopent-2-enecarboxylic acid pyrrolidine amide (0.17 g, 0.62 mmol) was oxidized according to procedure B at −20° C. The reaction mixture was washed with 5% NaOH. Purification by flash chromatography, yield 55 mg (0.20 mmol).

¹³C NMR: δ 24.42, 26.16, 27.77, 33.95, 45.86, 46.90, 49.41, 123.21, 133.96, 136.61, 144.16, 148.14, 150.14, 152.56, 172.49, 191.93.

ESI-MS: m/z 271 (M+H)⁺.

Anal. (C₁₆H₁₈N₂O₂.0.6 H₂O) calcd C: 68.36, H: 6.88, N: 9.96; found C: 68.70, H: 6.90, N: 9.60.

### DETERMINATION OF INHIBITORY EFFECT OF NOVEL COMPOUNDS ON PROLYL OLIGOPEPTIDASE ACTIVITY OF PIG BRAIN

The inhibitory effect of the novel compounds on POP activity of pig brain was determined with a method based on that described by Toide et al. (Toide, K, Iwamoto, Y., Fujiwara. T., Abe, H., J. Pharmacol. Exp. Ther., 1995, 274, 1370-1378) for the rat enzyme.

The whole pig brains, excluding cerebellum and most of the brain stem, of three pigs were placed in liquid nitrogen within 30 min from killing and stored at −80° C. until homogenized. The brains were homogenized with a glass-teflon homogenisator in 3 volumes (w/v) of ice-cold 0.1 M sodium-potassium phosphate buffer (pH 7.0) and the homogenates were centrifuged for 20 min at 4° C. at 10000 g. The supernatants were collected, pooled and stored in small aliquots at −80° C. until used. The supernatant was thawn in ice just before activity assay and diluted in a ratio 1:2 with homogenisation buffer (=enzyme preparation).

In the microplate assay procedure, 10 μl of enzyme preparation was preincubated with 460 μl of 0.1 M sodium-potassium phosphate buffer (pH 7.0) and 5 μl of a solution of novel compound dissolved in DMSO and diluted with 0.1 M sodium-potassium phosphate buffer at 30° C. for 30 min. The controls contained 10 μl enzyme preparation and 465 μl of 0.1 M sodium-potassium phosphate buffer (pH 7.0). The reaction was initiated by adding 25 μl of 4 mM Suc-Gly-Pro-AMC (AMC: 7-amido-4-methylcoumarin) dissolved in 0.1 M sodium-potassium phosphate buffer (pH 7.0), and the mixture was incubated at 30° C. for 60 min. The reaction was terminated by adding 500 μl of 1 M sodium acetate buffer (pH 4.2).

Formation of 7-amido-4-methylcoumarin was determined fluorometrically with microplate fluorescence reader (excitation at 360 nm and emission at 460 nm). The final concentration of novel compounds in the assay mixture varied from 10⁻¹² M to 10⁻⁴ M.

The prolyl oligopeptidase activity was calculated with the following formula in the presence of various concentrations of novel compounds. To reveal the inhibitory potency of the novel compound, activities (% of control) were plotted against the log concentration of the compound, and the IC₅₀ value was determined by non-linear regression utilizing GraphPad Prism software.
Activity (% of control)=a/b×100, where

a=fluorescence intensity in the presence of a novel compound

b=fluorescence intensity without a novel compound (control)

TABLE 1
Inhibition of pig brain prolyl oligopeptidase.

| Compound of | |
|---|---|
| example No. | IC₅₀ [nM] |
| 1 | 0.38 |
| 2 | 0.32 |
| 3 | 9 |
| 4 | 7.7 |
| 5 | 0.21 |
| 6 | 1.3 |
| 7 | 0.71 |
| 8 | 0.15 |
| 9 | 2.2 |
| 11 | 1.6 |
| 12 | 0.24 |
| 14 | 1.4 |
| 15 | 0.17 |
| 16 | 9.2 |

The novel compounds exhibit high inhibition potency against pig brain prolyl oligopeptidase. The results are summarized in Table 1.

Inhibitory Activity against Other Proline Specific Proteases

The novel compounds were tested for specificity of inhibitory activity against formation of 7-amido-4-methylcoumarin from specific substrates of other proline specific peptidases in the pig brain.

Determination of Inhibitory Effect of Novel Compounds on Dipeptidyl Peptidase II Activity of Pig Brain

By following the procedure for determination of inhibitory effect of novel compounds on prolyl oligopeptidase, but initiating the reaction by adding 25 μl of 0.4 mM H-Lys-Ala-AMC dissolved in 0.1 M sodium-potassium phosphate buffer (pH 7.0), and incubating the mixture at 30° C. for 30 min, the formation of 7-amido-4-methylcoumarin was determined. The dipeptidyl peptidase II inhibition was calculated with the following formula in the presence of a novel compound (10⁻⁶ M).
Percent inhibition (%)=(1−c/d)×100, where

c=fluorescence intensity in the presence of novel compound

d=fluorescence intensity without novel compound (control)

The novel compounds did not exhibit any inhibitory effect against pig brain dipeptidyl peptidase II.

Determination of Inhibitory Effect of Novel Compounds on Dipeptidyl Eptidase IV Activity of Pig Brain

By following the procedure for determination of inhibitory effect of novel compounds on prolyl oligopeptidase, but initiating the reaction by adding 25 μl of 2 mM H-Gly-Pro-AMC dissolved in 0.1 M sodium-potassium phosphate buffer (pH 7.0), the formation of 7-amido-4-methylcoumarin was determined. The dipeptidyl peptidase IV inhibition was calculated with the formula described above in the presence of a novel compound (10⁻⁶ M).

The novel compounds did not exhibit any inhibitory effect against pig brain dipeptidyl peptidase IV.

1. A compound of formula (I) 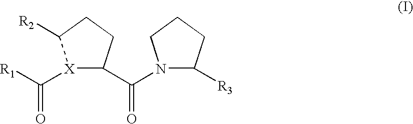
wherein in the formula, X is N or C;
the dotted line represents a single or a double bond;
R₁ is:
a straight or branched alkyl chain having 1 to 10 carbon atoms unsubstituted or substituted with 1 to 3 substituent(s) each independently being COOR⁴, COR⁴, CR⁴(OR⁵)₂, COCH₂OR⁶, cyano, hydroxy, oxo, halogen, lower alkoxy, aryl, aryloxy, aryl lower alkoxy, nitro, amino, lower alkyl amino, aryl amino, aryl lower alkyl amino, cycloalkyl or heterocycle, wherein R⁴ is H, lower alkyl, lower alkenyl, cycloalkyl, cycloalkenyl, heterocycle, aryl or aralkyl, R⁵ is lower alkyl, lower alkenyl, cycloalkyl, cycloalkenyl, aryl or aralkyl and R⁶ is H, lower alkyl, lower acyl or halogen,
a straight or branched alkenyl chain having 2 to 10 carbon atoms unsubstituted or substituted with 1 to 3 substituent(s) as defined for the alkyl group above,
a 3 to 7 membered, saturated or unsaturated, carbocyclic ring unsubstituted or substituted with 1 to 3 substituent(s) each independently being lower alkyl or as defined for the alkyl group above,
a 3 to 7 membered, saturated or unsaturated, heterocyclic ring unsubstituted or substituted with 1 to 3 substituent(s) each independently being lower alkyl or as defined for the alkyl group above,
a substituted or unsubstituted alkyl or alkenyl group as defined above incorporating as a group member a substituted or unsubstituted carbocyclic ring or a heterocyclic ring as defined above,
hydroxy, lower alkoxy, aryloxy, aryl lower alkoxy, amino, amino lower alkyl, lower alkyl amino, aryl amino or aryl lower alkyl amino, wherein the said alkyl, aryl, or amino subgroups are unsubstituted or substituted with 1 to 3 substituent(s) each independently being lower alkyl or as defined for the alkyl group above;
R₂ is:
H,
a straight or branched alkyl chain having 1 to 10 carbon atoms unsubstituted or substituted with 1 to 3 substituent(s) each independently being hydroxy, oxo, lower alkoxy, amino, lower alkyl amino, halogen, carboxyl, or lower acyl,
a straight or branched alkenyl chain having 2 to 10 carbon atoms unsubstituted or substituted with 1 to 3 substituent(s) as defined for the alkyl group, in the meaning of R₂, above,
or a straight or branched alkynyl chain having 2 to 10 carbon atoms unsubstituted or substituted with 1 to 3 substituent(s) as defined for the alkyl group, in the meaning of R₂, above;
R₃ is:
H, cyano, hydroxy, oxo, halogen, lower alkyl, lower alkoxy, aryl, aryloxy, aryl lower alkoxy, amino, lower alkyl amino, aryl amino, aryl lower alkyl amino, cycloalkyl or heterocycle, wherein the said alkyl subgroups are unsubstituted or substituted with 1 to 3 substituent(s) as defined for the alkyl group, in the meaning of R₁, above,
or R₃ is COOR⁴, COR⁴, CR⁴(OR⁵)₂ or COCH₂OR⁶, wherein R⁴ is H, lower alkyl, alkenyl, cycloalkyl, cycloalkenyl, heterocycle, aryl, amino, lower alkyl amino, aryl amino or lower alkyl amino, wherein the said lower alkyl is unsubstituted or substituted with 1 or 2 substituent(s) each independently being cyano, hydroxy, oxo, halogen, lower alkoxy, aryl, aryloxy, aryl lower alkoxy, amino, lower alkyl amino, aryl amino, aryl lower alkyl amino, cycloalkyl or heterocycle, R⁵ is lower alkyl, lower alkenyl, cycloalkyl, cycloalkenyl, aryl or aralkyl and R⁶ is lower acyl or halogen;
or a pharmaceutically acceptable salt or ester thereof,
provided, that
a) when X is N, the dotted line represents a single bond and R₂ is not H;
b) when X is C, the dotted line represents a double bond and R₂ is H;
c) the compound is not 5-ethoxycarbonyl-N-benzyloxycarbonyl-2-[(2′-(S)-benzylcarbonyl)-1′-pyrrolidinylcarbonyl]pyrrolidine or 1,2-pyrrolidinedicarboxylic acid, 5-(1-pyrrolidinylcarbonyl)-,1-(phenylmethyl) ester.

2. A compound according to claim 1, wherein
X is N;
the dotted line represents a single bond;
R₁ is:
a straight or branched alkyl chain having 1 to 10 carbon atoms unsubstituted or substituted with 1 to 3 substituent(s) each independently being COOR⁴, COR⁴, CR⁴(OR⁵)₂, COCH₂OR , cyano, hydroxy, oxo, halogen, lower alkoxy, aryl, aryloxy, aryl lower alkoxy, nitro, amino, lower alkyl amino, aryl amino, aryl lower alkyl amino, cycloalkyl or heterocycle, wherein R⁴ is H, lower alkyl, lower alkenyl, cycloalkyl, cycloalkenyl, heterocycle, aryl or aralkyl, R⁵ is lower alkyl, lower alkenyl, cycloalkyl, cycloalkenyl, aryl or aralkyl and R⁶ is H, lower alkyl, lower acyl or halogen,
a straight or branched alkenyl chain having 2 to 10 carbon atoms unsubstituted or substituted with 1 to 3 substituent(s) as defined for the alkyl group above,
a 3 to 7 membered, saturated or unsaturated, carbocyclic ring unsubstituted or substituted with 1 to 3 substituent(s) each independently being lower alkyl or as defined for the alkyl group above,
a 3 to 7 membered, saturated or unsaturated, heterocyclic ring unsubstituted or substituted with 1 to 3 substituent(s) each independently being lower alkyl or as defined for the alkyl group above,
a substituted or unsubstituted alkyl or alkenyl group as defined above incorporating as a group member a substituted or unsubstituted carbocyclic ring or a heterocyclic ring as defined above,
hydroxy, lower alkoxy, aryloxy, aryl lower alkoxy, amino, amino lower alkyl, lower alkyl amino, aryl amino or aryl lower alkyl amino, wherein the said alkyl, aryl or amino subgroups are unsubstituted or substituted with 1 to 3 substituent(s) each independently being lower alkyl or as defined for the alkyl group above;
R₂ is:
a straight or branched alkyl chain having 1 to 10 carbon atoms unsubstituted or substituted with 1 to 3 substituent(s) each independently being hydroxy, oxo, lower alkoxy, amino, lower alkyl amino, halogen, carboxyl or lower acyl,
a straight or branched alkenyl chain having 2 to 10 carbon atoms unsubstituted or substituted with 1 to 3 substituent(s) as defined for the alkyl group, in the meaning of R₂, above,
or a straight or branched alkynyl chain having 2 to 10 carbon atoms unsubstituted or substituted with 1 to 3 substituent(s) as defined for the alkyl group, in the meaning of R₂, above;
R₃ is:
H, cyano, hydroxy, oxo, halogen, lower alkyl, lower alkoxy, aryl, aryloxy, aryl lower alkoxy, amino, lower alkyl amino, aryl amino, aryl lower alkyl amino, cycloalkyl or heterocycle, wherein the said alkyl subgroups are unsubstituted or substituted with 1 to 3 substituent(s) as defined for the alkyl group, in the meaning of R₁, above,
or R₃ is COOR⁴, COR⁴, CR⁴(OR⁵)₂ or COCH₂OR⁶, wherein R⁴ is H, lower alkyl, lower alkenyl, cycloalkyl, cycloalkenyl, heterocycle, aryl, amino, lower alkyl amino, aryl amino or lower alkyl amino, wherein the said lower alkyl is unsubstituted or substituted with 1 or 2 substituent(s) each independently being cyano, hydroxy, oxo, halogen, lower alkoxy, aryl, aryloxy, aryl lower alkoxy, amino, lower alkyl amino, aryl amino, aryl lower alkyl amino, cycloalkyl or heterocycle, R⁵ is lower alkyl, lower alkenyl, cycloalkyl, cycloalkenyl, aryl or aralkyl and R⁶ is lower acyl or halogen, or a pharmaceutically acceptable salt or ester thereof.

3. A compound according to claim 2, wherein
R₁ is
a straight or branched alkyl chain having 1 to 5 carbon atoms unsubstituted or substituted with 1 or 2 substituent(s) each independently being hydroxy, halogen, lower alkoxy, aryl, aryloxy, aryl lower alkoxy, amino, lower alkyl amino, aryl amino, aryl lower alkyl amino, cycloalkyl or heterocycle,
a 3 to 7 membered, saturated or unsaturated, carbocyclic ring unsubstituted or substituted with 1 or 2 substituent(s) each independently being lower alkyl or as defined for the alkyl group above,
a 3 to 7 membered, saturated or unsaturated, heterocyclic ring unsubstituted or substituted with 1 or 2 substituent(s) each independently being lower alkyl or as defined for the alkyl group above,
a substituted or unsubstituted alkyl or alkenyl group as defined above incorporating as a group member a substituted or unsubstituted carbocyclic ring or a heterocyclic ring as defined above,
hydroxy, lower alkoxy, aryloxy, aryl lower alkoxy, amino, amino lower alkyl, lower alkyl amino, aryl amino or aryl lower alkyl amino, wherein the said alkyl, aryl or amino subgroups are unsubstituted or substituted with 1 to 3 substituent(s) each independently being lower alkyl or as defined for the alkyl group above;
R₂ is
a straight or branched alkyl chain having 1 to 5 carbon atoms unsubstituted or substituted with 1 or 2 substituent(s) each independently being hydroxy, oxo, lower alkoxy, amino, lower alkyl amino, halogen, carboxyl, or lower acyl;
R₃ is:
H, cyano or COR⁴, wherein R⁴ is H, lower alkyl, cycloalkyl, cycloalkenyl, heterocycle or aryl, wherein the said lower alkyl is unsubstituted or substituted with 1 or 2 substituent(s) each independently being hydroxy, oxo, halogen, lower alkoxy, aryl, aryloxy, aryl lower alkoxy, cycloalkyl or heterocycle.

4. A compound according to claim 2, wherein
R₁ is
a straight alkyl chain having 1 to 3 carbon atoms unsubstituted or substituted with 1 or 2 substituent(s) each independently being aryl, aryloxy, aryl lower alkoxy, lower alkyl amino, aryl amino, aryl lower alkyl amino, cycloalkyl or heterocycle,
a 3 to 7 membered, saturated or unsaturated, unsubstituted heterocyclic ring, lower alkoxy, lower alkyl amino, aryl amino or aryl lower alkyl amino;
R₂ is a straight or branched unsubstituted alkyl chain having 1 to 4 carbon atoms;
R₃ is:
H, cyano or COR⁴, wherein R⁴ is H or lower alkyl, wherein the said lower alkyl is unsubstituted or substituted with hydroxy.

5. A compound according to claim 1, wherein
X is C;
the dotted line represents a double bond;
R₁ is:
a straight or branched alkyl chain having 1 to 10 carbon atoms unsubstituted or substituted with 1 to 3 substituent(s) each independently being COOR⁴, COR⁴, CR⁴(OR⁵)₂, COCH₂OR⁶, cyano, hydroxy, oxo, halogen, lower alkoxy, aryl, aryloxy, aryl lower alkoxy, nitro, amino, lower alkyl amino, aryl amino, aryl lower alkyl amino, cycloalkyl or heterocycle, wherein R⁴ is H, lower alkyl, lower alkenyl, cycloalkyl, cycloalkenyl, heterocycle, aryl or aralkyl, R⁵ is lower alkyl, lower alkenyl, cycloalkyl, cycloalkenyl, aryl or aralkyl and R⁶ is H, lower alkyl, lower acyl or halogen,
a straight or branched alkenyl chain having 2 to 10 carbon atoms unsubstituted or substituted with 1 to 3 substituent(s) as defined for the alkyl group above,
a 3 to 7 membered, saturated or unsaturated, carbocyclic ring unsubstituted or substituted with 1 to 3 substituent(s) each independently being lower alkyl or as defined for the alkyl group above,
a 3 to 7 membered, saturated or unsaturated, heterocyclic ring unsubstituted or substituted with 1 to 3 substituent(s) each independently being lower alkyl or as defined for the alkyl group above,
a substituted or unsubstituted alkyl or alkenyl group as defined above incorporating as a group member a substituted or unsubstituted carbocyclic ring or a heterocyclic ring as defined above,
hydroxy, lower alkoxy, aryloxy, aryl lower alkoxy, amino, amino lower alkyl, lower alkyl amino, aryl amino or aryl lower alkyl amino, wherein the said alkyl, aryl or amino subgroups are unsubstituted or substituted with 1 to 3 substituent(s) each independently being lower alkyl or as defined for the alkyl group above;
R₂ is H;
R₃ is:
H, cyano, hydroxy, oxo, halogen, lower alkyl, lower alkoxy, aryl, aryloxy, aryl lower alkoxy, amino, lower alkyl amino, aryl amino, aryl lower alkyl amino, cycloalkyl or heterocycle, wherein the said alkyl subgroups are unsubstituted or substituted with 1 to 3 substituent(s) as defined for the alkyl group, in the meaning of R₁, above,
or R₃ is COOR⁴, COR⁴, CR⁴(OR⁵)₂ or COCH₂OR , wherein R⁴ is H, lower alkyl, lower alkenyl, cycloalkyl, cycloalkenyl, heterocycle, aryl, amino, lower alkyl amino, aryl amino or lower alkyl amino, wherein the said lower alkyl is unsubstituted or substituted with 1 or 2 substituent(s) each independently being cyano, hydroxy, oxo, halogen, lower alkoxy, aryl, aryloxy, aryl lower alkoxy, amino, lower alkyl amino, aryl amino, aryl lower alkyl amino, cycloalkyl or heterocycle, R⁵ is lower alkyl, lower alkenyl, cycloalkyl, cycloalkenyl, aryl or aralkyl and R⁶ is lower acyl or halogen.

6. A compound according to claim 5, wherein
R₁ is:
a straight or branched alkyl chain having 1 to 5 carbon atoms unsubstituted or substituted with 1 or 2 substituent(s) each independently being hydroxy, halogen, lower alkoxy, aryl, aryloxy, aryl lower alkoxy, amino, lower alkyl amino, aryl amino, aryl lower alkyl amino, cycloalkyl or heterocycle,
a 3 to 7 membered, saturated or unsaturated, carbocyclic ring unsubstituted or substituted with 1 or 2 substituent(s) each independently being lower alkyl or as defined for the alkyl group above,
a 3 to 7 membered, saturated or unsaturated, heterocyclic ring unsubstituted or substituted with 1 or 2 substituent(s) each independently being lower alkyl or as defined for the alkyl group above,
a substituted or unsubstituted alkyl or alkenyl group as defined above incorporating as a group member a substituted or unsubstituted carbocyclic ring or a heterocyclic ring as defined above,
hydroxy, lower alkoxy, aryloxy, aryl lower alkoxy, amino, amino lower alkyl, lower alkyl amino, aryl amino or aryl lower alkyl amino, wherein the said alkyl, aryl, or amino subgroups are unsubstituted or substituted with 1 to 3 substituent(s) each independently being lower alkyl or as defined for the alkyl group above;
R₃ is:
H, cyano or COR⁴, wherein R⁴ is H, lower alkyl, cycloalkyl, cycloalkenyl, heterocycle or aryl, wherein the said lower alkyl is unsubstituted or substituted with 1 or 2 substituent(s) each independently being hydroxy, oxo, halogen, lower alkoxy, aryl, aryloxy, aryl lower alkoxy, cycloalkyl or heterocycle.

7. A compound according to claim 5, wherein
R₁ is
a straight or branched alkyl chain having 1 to 3 carbon atoms unsubstituted or substituted with 1 or 2 substituent(s) each independently being, aryl, aryloxy, aryl lower alkoxy, lower alkyl amino, aryl amino, aryl lower alkyl amino, cycloalkyl, or heterocycle,
a 3 to 7 membered, saturated or unsaturated, unsubstituted heterocyclic ring, lower alkoxy, amino lower alkyl, lower alkyl amino, aryl amino or aryl lower alkyl amino, wherein the amino subgroups are unsubstituted or substituted with lower alkyl;
R₃ is:
H, cyano or COR⁴, wherein R⁴ is H or lower alkyl, wherein the said lower alkyl is unsubstituted or substituted with hydroxy.

8. A pharmaceutical composition comprising at least one compound according to claim 1 and a pharmaceutically acceptable diluent, carrier and/or excipient.

9-12. (canceled)

13. A method for the treatment of a disease or for the enhancement of a condition where prolyl oligopeptidase inhibitors are indicated to be useful, which comprises administering to a subject in need of the treatment or enhancement an effective amount of at least one compound according to claim 1.

14. The method according to claim 13, which comprises treating a neurodegenerative disease, and/or improving learning and memory functions.

15. The method according to claim 14, wherein the neurodegenerative disease is Alzheimer's disease or senile dementia.
